(19) United States
(12) Patent Application Publication
(10) Pub. No.: US 2002/0181658 A1
(43) Pub. Date: Dec. 5, 2002

(54) Comfort accessory for dental X-rays

(76) Inventor: Leslie A. Garcia, Lakeworth, FL (US)

Correspondence Address: Donald D. Mon, 750 East Green Street, Suite 303, Pasadena, CA, 91101, US

(21) Appl. No.: 09/874,443

(22) Filed: Jun. 4, 2001

(51) Int. Cl. A61B 6/14; G03B 42/04

(52) U.S. Cl. 378/169; 378/168

(57) ABSTRACT

A cushion attached to the edge of a hard film container for dental X-rays to reduce the pain of contact with dental tissue. The cushion is a foam, and flavoring can be applied to it.

### FIELD OF THE INVENTION

Cushions for attachment to the edges of a hard dental X-ray film container.

### BACKGROUND OF THE INVENTION

Every person who has had a dental X-ray will recall the unpleasant pain that occurs when the dentist instructs the patient to bite down onto the container of an X-ray film. While the pain is quite temporary, it occurs during a period of uncertainty for the patient—a time when any additional discomfort or distraction is best avoided. The situation is often bad enough already.

The problem cannot be solved by altering the container. In order to obtain a suitable image, the placement end shape of the film must be kept within close limits. The attractive accommodation of a fully flexible and comfortable basic structure to contact the dental tissue is not available and the patient must simply accommodation himself to it. After many decades, the uncomfortable conventional arrangement is the accepted norm.

It is an object of this invention to provide a compressible means to mitigate the painful contact, but without deforming the film to an unacceptable extent.

It is an optional object of this invention to provide the compressible means with flavoring material to add the element of a pleasant surprise.

### BRIEF DESCRIPTION OF THE INVENTION

This invention is used in combination with the conventional container for a dental X-ray film. Such a container is quite stiff and deforms to a limited extent only when pressed strongly against dental tissue adjacent to the teeth being imaged. Such dental tissue can include gums, their adjacent jaw and skull tissue, and checks.

According to this invention, a compressible foam cushion is fitted to the edge of a container for the film, at least in regions where, depending on the location of tooth to be imaged, the cushion will provide a soft contact with the tissue and prevent hard contact with the container. This involves only a small attendant and acceptable spacing, while still reducing or eliminating a painful contact with the tissue.

According to a preferred but optional feature of the invention, the cushion is provided as discrete lengths of U-shaped foam material attached to the container at suitable locations. It desired, it may be made so as to be removable. It can be removed after the film is removed it the container is to be re-used.

According to yet another feature of the invention, the cushion is made from a foam whose basic structure is substantially transparent to X-ray, so it can not interfere with the exposure or processing of the film.

As an optional feature of the invention, flavoring material may be added to the foam,

The above and other features of this invention will be fully understood from the following detailed description and the accompanying drawings, in which;

### BRIEF DESCRIPTION OF THE DRAWINGS

FIG. 1 is a side view of a film container incorporating the invention:

FIG. 2 is a cross-section taken at line 2-2 in FIG. 1;

FIG. 3 is a side view of a strip of material suitable for forming the cushions; and

FIG. 4 is a side view of FIG. 3.

### DETAILED DESCRIPTION OF THE INVENTION

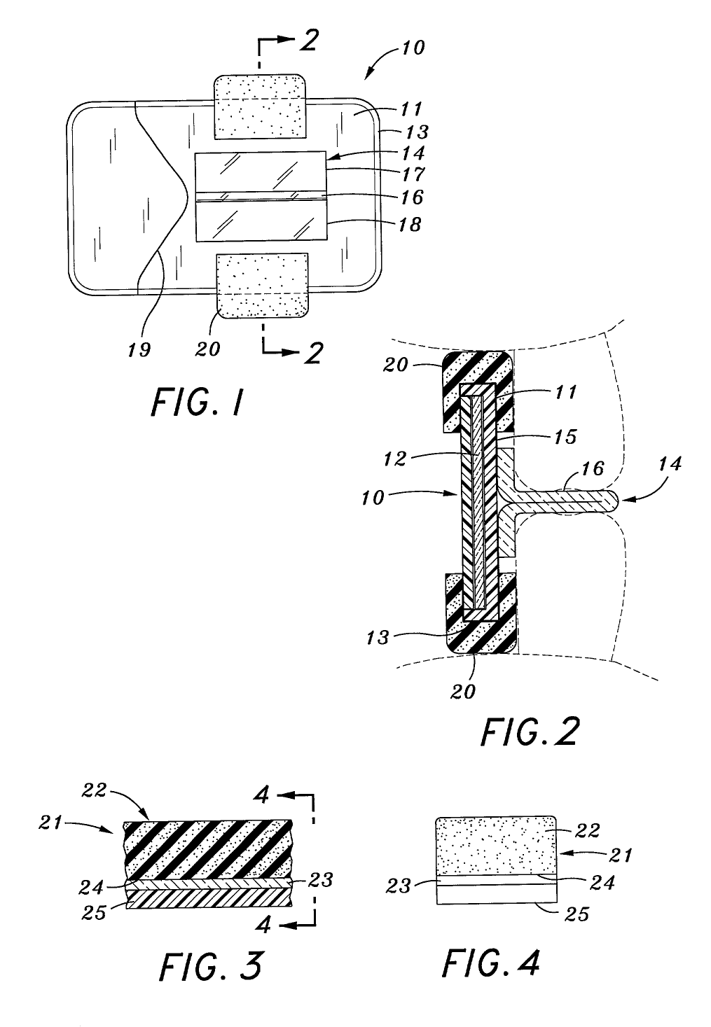

A film container 10 with which the invention to used, is shown in FIGS. 1 and 2. It is generally flat and rectangular, although it can have other specialized shapes such as for children and for anterior views. Its cover 11 holds an X-ray film 12. The cover is transparent to X-rays.

Importantly to this invention it has a peripheral edge 13 which is quite hard and only stiffly deformable so the film will be held in a correct shape.

A tab 14 attached to the cover extends from face 15 of the cover. It is a piece of paper, adhesively coated on one side, folded to form a flange 16, and skirts 17, 18 to be attached to the container. It also is transparent to X-rays. The patient is instructed to bite on this tab while the cover is pressed against adjacent tissue, and hold it that way while the X-ray is taken. This is the source of a continuing pain and discomfort, and at least an unwelcome distraction.

Users of these containers will recognize the peel-back fold 19 which closes the film inside the container, along with the customary shields. It is generally formed on the back side of the container.

The object of this invention is to eliminate the direct contact of the container and the tissue. For this purpose cushions 20 are shown attached to the container at its edge. If desired a cushion could extend entirely around the container. However, in practical office usage it will be used in smaller pieces, either in the form of precut pieces, or supplied from a coil of flat length material. The desired lengths can be cut from this raw material.

FIGS. 1 and 2 show the cushions already in place, removably adhered to the container. FIGS. 3 and 4 show a piece 21 of cushion material comprised of a layer 22 of compressible open or closed call foam which is transparent to X-rays. It includes a layer of adhesive 23 on one face 24, and a removable cover sheet 25 over the adhesive to protect the adhesive until the foam is to be attached to the container. Sheet 25 is readily peeled off, and the foam may readily be attached to the container. Foams stronger then the adhesive, and cover sheets readily releasable from adhesives are well-known in the art. If preferred, the cushions will not be removed when the container itself is to be discarded.

As to the foam layer, its thickness should be minimized so that while it spaces some or all of the edge from the tissue, it is not so large a spacing as to interfere with proper positioning of the film, even when fully compressed. Accordingly, a soft layer of foam compressible to so little as 10% of its volume may be used. Any suitable foam material usually an organic plastic foam can be used.

Flavoring substances—peppermint, bubble gum, cherry, and any other agreeable flavor can be applied to the custom material to add the pleasant surprise of an agreeable taste,

In use, before the X-ray is to be taken, the technician either selects or cuts a suitable length of foam peels the cover sheet off, and folds the foam over the edge with the adhesive against the container. The X-ray is taken routinely, and the foam can be removed if the container is to be reused, or left on the container to be thrown away with it if the container is to be discarded. Removability may not always be required.

This invention is not to be limited by the embodiments shown in the drawings and described in the description, which are given by way of example and not of limitation, but only in accordance with the scope of the appended claims.

I claim:

1. In combination:
a container containing an X-ray film to be placed in the mouth of a dental patient, said container having a substantial lateral extent and a peripheral edge, said container, and specially said edge being hard, said contained being intended to contact tissue adjacent to teeth when a tab on said cover is clenched between the teeth; and
a cushion of compressible foam adhered to said cover and overlapping at least a portion of said edge, whereby to form the first contact between the portion of said edge occupied by said cushion and said tissue, thereby to reduce pain which would otherwise have been caused by direct contact of that portion of the edge and the tissue.

2. A combination according to claim 1 in which said cushion is a cellular foam.

3. A combination to claim 2 in which said foam is transparent to X-rays.

4. A combination according to claim 2 in which a flavoring substance is applied to the foam.

5. A combination according to claim 1 which a layer of adhesive on said cushion adheres the cushion to the container.

6. A combination according to claim 5 in which said adhesive is removable from the container.

7. A cushion for reducing the discomfort of a dental patient about to undergo the procedure of a dental X-ray, said procedure including the use of a container containing an X-ray film to be placed in the mouth of a dental patient, said container having a substantial lateral extent and a peripheral edge, said container, and especially said edge being hard, said container being intended to contact tissue adjacent to teeth when a tab on said cover is clenched between the teeth, said cushion comprising a layer of compressible foam and a layer of adhesive for being adhered to said cover and overlapping at least a portion of said edge, whereby to form the first contact between the portion of said edge occupied by said cushion and said tissue, thereby to reduce pain which would otherwise have been caused by direct contact of that portion of the edge and the tissue.

8. A cushion according to claim 7 in which a flavoring substance is applied to the foam.

9. A cushion according to claim 7 in which said adhesive is releasable from the container.
